(19) United States
(12) Patent Application Publication
(10) Pub. No.: US 2025/0275791 A1
(43) Pub. Date: Sep. 4, 2025

(54) PEDICLE BASED RETRACTOR WITH SCREW GATE

(71) Applicant: Zavation Medical Products, LLC, Flowood, MS (US)

(72) Inventors: Graham Carr Calvert, Ridgeland, MS (US); John Lamar Wilson, Brandon, MS (US); John Lawrence Walker, JR., Madison, MS (US); Katie Lynn Motley, Flowood, MS (US)

(21) Appl. No.: 18/591,533

(22) Filed: Feb. 29, 2024

Related U.S. Application Data
(60) Provisional application No. 63/442,032, filed on Jan. 30, 2023.

(51) Int. Cl. A61B 17/70; A61B 17/02

(52) U.S. Cl. CPC A61B 17/7085; A61B 17/0206; A61B 17/0218; A61B 17/025; A61B 2017/0256

(57) ABSTRACT

A retractor blade assembly having a retractor blade having a track extending in a longitudinal direction along on a face of the retractor blade, a screw entry at a bottom of the retractor blade comprising a window through which a bone screw is insertable, and a screw gate having a protrusion on a back surface which engages the track on the retractor blade. Once the bone screw is placed through the screw entry, the screw gate lowers until the screw gate blocks the window.

### CROSS REFERENCE TO RELATED APPLICATIONS

This application is related to and claims priority to U.S. Provisional Patent Application Ser. No. 63/442,032 filed Jan. 30, 2023 entitled PEDICLE BASED RETRACTOR WITH SCREW GATE, the entire contents of which are incorporated herein by reference.

### BACKGROUND OF THE INVENTION

### Field of the Invention

The present invention relates to retractor devices used during spinal operation procedures.

### Description of the Related Art

Disorders of the bone include disorders such as for example degenerative disc disease, disc herniation, osteoporosis, spondylolisthesis, stenosis, scoliosis (and other curvature abnormalities), kyphosis, tumor, fracture, arthritis, calcification, etc. Such disorders may result from factors including trauma, disease and degenerative conditions caused by injury and aging. Bone disorders typically result in symptoms including pain, nerve damage, and partial or complete loss of mobility. Additionally, severe pain and discomfort can occur due to the pressure exerted by bones on nerves.

A spinal disc can become damaged as a result of degeneration, dysfunction, disease and/or trauma. Conservative treatment can include non-operative treatment through exercise and/or pain relievers to deal with the pain. Operative treatment options include disc removal and replacement using an interbody spacers such as anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF).

The spacers are placed in the interdiscal space between adjacent vertebrae of the spine, resulting in spinal fusion of the adjacent vertebra wherein two or more vertebrae are joined together (fused) by way of interbody spacers, sometimes with bone grafting, to form a single bone. Typically, interbody fusion requires surgical removal of all or a portion of the intervertebral disc. After removal of the intervertebral disc, the interbody spacer is inserted in the space between the adjacent vertebrae.

There has been considerable development of retractors and retractor systems that are adapted for use in less invasive procedures. Many of the recent developments are based on traditional types of surgical retractors for open procedures, predominantly table-mounted devices of various designs. These devices tend to be cumbersome and are not well adapted for use in small incisions. Standard hand-held surgical retractors are well known in the prior art and can be modified to fit the contours of these small incisions, but they require manual manipulation to maintain a desired placement, thereby occupying one hand of the physician or requiring another person to assist the physician during the procedure. Typical retractors are also positioned into the soft tissue and are levered back to hold the wound open, frequently requiring re-positioning if they dislodge, obstruct the physician's view, or interfere with access to the surgical site.

In recent years, minimally invasive surgical approaches have been applied to orthopedic surgery and more recently to spine surgery, such as instrumented fusions involving one or more vertebral bodies. Unlike minimally invasive procedures such as arthroscopic knee surgery or gallbladder surgery where the affected area is contained within a small region of the body, spinal fusion surgery typically encompasses a considerably larger region of the patient's body. In addition, arthroscopic surgery and laparoscopic surgery permit the introduction of fluid (i.e. liquid or gas) for distending tissue and creating working space for the surgeon. Surgery on the spine does not involve a capsule or space that can be so distended, instead involving multiple layers of soft tissue, bone, ligaments, and nerves. For these reasons, the idea of performing a minimally invasive procedure on the spine has only recently been approached.

By way of example, in a typical spine fusion at least two vertebral bodies are rigidly connected using screws implanted into the respective vertebral bodies with a solid metal rod spanning the distance between the screws. This procedure is not generally conducive to a minimally invasive approach. The insertion of pedicle or facet screws is relatively straightforward and can be accomplished through a minimal incision. The difficulty arises upon the introduction of a length of rod into a very small incision with extremely limited access and visibility. A single level fusion may require a 30-40 mm rod to be introduced into a 1 cm incision and a multilevel fusion may require a rod several inches long to fit into a 1 cm incision. For this reason, it is important that the minimal incision be maintained in an open and accessible condition (i.e. as wide as practicable) for introduction of the rod.

Minimally invasive surgery offers significant advantages over conventional open surgery. First, the skin incision and subsequent scar are significantly smaller. By using more than one small incision rather than one large incision, the need for extensive tissue and muscle retraction may be greatly reduced. This leads to significantly reduced post-operative pain, a shorter hospital stay, and a faster overall recovery.

Medtronic Sofamor Danek's SEXTANT® is a minimally invasive device used for screw and rod insertion. Its shortcomings lie with how complicated the system is to use and the requirement for an additional incision for rod introduction. This system also requires that the guidance devices be rigidly fixed to the pedicle screw head in order to maintain instrument alignment and to prevent cross-threading of the setscrew. For these reasons, the surgeon cannot access the surrounding anatomy for complete preparation of the field. Nor does SEXTANT® allow for any variation in the procedure, if need be.

Depuy Spine's VIPER™ system is another minimally invasive implant and technique recommended for one or two level spine fusions. This system is less complicated than the SEXTANT® only requiring two incisions for a unilateral, one-level fusion, but it is limited in the same way as the SEXTANT® because it also requires the instrumentation to be rigidly fixed to the pedicle screw.

Spinal Concept's PATHFINDER® and NuVasive's SPHERX® spinal system (as disclosed in U.S. Pat. No. 6,802,844), are marketed as “minimally disruptive” spine fusion implants and procedures. While they have advantages over a general “open” procedure, they do not provide all of the advantages of a truly minimally invasive approach. Their characterization as “minimally open” procedures is a result of the inherent difficulty of introducing a rod in a minimally invasive spinal procedure. In order to introduce a rod long enough to accomplish a single level fusion, these systems describe an incision long enough to accept such a rod, thereby undermining the advantages of a minimally invasive approach.

The following provides a non-exhaustive list of implant devices and systems known in the art.

U.S. Pat. No. 10,758,370 (the entire contents of which are incorporated herein by reference) describes implant assemblies, systems, and methods for stabilizing a joint between a superior vertebra and an inferior vertebra. These systems included a plate member coupled to an interbody spacer with at least one fastener that extends superiorly or inferiorly from the implant assembly to anchor within a vertebral body and stabilize the joint. In the '370 patent, inserters were used to facilitate fixation of the various implant assemblies within the intervertebral space of the joint between the superior vertebra and the inferior vertebra.

U.S. Pat. Appl. Publ. No. 20210322185 (the entire contents of which are incorporated herein by reference) describes interbody fusion devices including deployable fixation members. The implant in the '185 publication included a spacer, optionally, an end member coupled to the spacer, and one or more fixation members configured to extend into adjacent vertebrae. The fixation members included screws, nails, shims, tangs, spikes, staples, pins, blades, fins, or the like, and combinations thereof.

U.S. Pat. No. 8,435,269 (the entire contents of which are incorporated herein by reference) describes instruments and methods for creating an operative corridor to the spine and performing a spinal fusion, the instruments including a retractor assembly with a plurality of retractor blades, at least two pedicle screws, and a connecting rod.

U.S. Pat. No. 11,154,288 (the entire contents of which are incorporated herein by reference) describes implants, instruments, and methods for performing surgical procedures on the spine, including one or more of creating an operative corridor to the spine, delivering implants to the spine, fusing one or more segments of the spine and fixing one or more segments of the spine

U.S. Pat. Appl. Publ. No. 20200297511 (the entire contents of which are incorporated herein by reference) describes implantable systems, devices and related methods pertaining to spinal surgery. In the '511 publication, a low profile frame and spacer system was used for inserting into a disc space. The frame could receive different fixation devices, including threaded and non-threaded fixation devices.

U.S. Pat. No. 9,795,370 (the entire contents of which are incorporated herein by reference) describes a surgical retractor and related methods for providing access to a surgical target site for the purpose performing minimally invasive spinal fusion across one or more segments of the spinal column.

U.S. Pat. No. 8,974,381 (the entire contents of which are incorporated herein by reference) describes an anterior cervical retractor comprising a first medial-lateral retractor body having a base arm and a moving arm and a pair of retractor blades. The retractor blades may be side loading or top loading. The cervical retractor comprises a second cranial-caudal retractor body having a pair of moving arms and a pair of retractor blades.

U.S. Pat. No. 11,246,715 (the entire contents of which are incorporated herein by reference) describes anchoring devices, anchoring systems for intervertebral implants, intervertebral implants, and instruments and methods for implanting implants. In the '715 patent, an anchoring device having a body comprising at least one curved plate elongated along a longitudinal axis was designed to be inserted through a passage crossing at least a part of implant, in order to penetrate into at least one vertebral endplate and attach implant onto this vertebral endplate.

U.S. Pat. No. 8,114,016 (the entire contents of which are incorporated herein by reference) describes a reconfigurable modular retractor system including a set of interchangeable body modules, the body modules including mechanisms for forming retractors and slot mechanisms for connecting blades, and blades including locking mechanisms for locking inside the slot mechanisms. The '016 patent describes a method of using the retractor system by forming a retractor by using at least one body module, attaching blades to the body module, inserting the retractor into a surgical area, retracting tissue, removing retraction from the tissue, and removing the retractor.

U.S. Pat. No. 2012/0296171 (the entire contents of which are incorporated herein by reference) describes implants, instruments, and methods for performing surgical procedures on the spine, including one or more of creating an operative corridor to the spine, delivering implants to the spine, fusing one or more segments of the spine, and fixing one or more segments of the spine.

U.S. Pat. No. 8,414,625 (the entire contents of which are incorporated herein by reference) describes a retraction device comprises at least one retracting blade and a pedicle screw. The retracting blade has a distal end having at least one projection with a passage for engaging a retention pin. The pedicle screw has a threaded body and a movable head. The movable head has at least one recess with a channel that can be aligned with the passage of the projection.

U.S. Pat. No. 10,660,628 (the entire contents of which are incorporated herein by reference) describes a surgical retractor and related methods for providing access to a surgical target site for the purpose performing minimally invasive spinal fusion across one or more segments of the spinal column.

### SUMMARY

In one embodiment, there is provided a retractor blade assembly having a retractor blade having a track extending in a longitudinal direction along on a face of the retractor blade, a screw entry at a bottom of the retractor blade comprising a window through which a bone screw is insertable, and a screw gate having a protrusion on a back surface which engages the track on the retractor blade. Once the bone screw is placed through the screw entry, the screw gate lowers until the screw gate blocks the window.

In one embodiment, there is provided a method for connecting a retractor blade to a bone screw. The method attaches the bone screw to a bony body, inserts the bone screw through a window at a base of the retractor bade, couples a screw gate to a face of the retractor blade, and translates the screw gate along a track extending longitudinally along a length of the retractor blade to a position where the screw gat blocks the window.

It is to be understood that both the foregoing general description of the invention and the following detailed description are exemplary, but are not restrictive of the invention.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawing.

FIG. 1 is a diagram depicting the insertion needle approach to and entry into the pedicle bone facet.

FIG. 2 is a diagram depicting the placement of a dilator over a K-wire.

FIG. 3 is a diagram depicting a cannulated tap inserted into the pedicle canal.

FIG. 4 is a schematic depicting a screwdriver guide assembly according to one embodiment of the present invention.

FIG. 5 is a schematic depicting a retractor blade according to one embodiment of the present invention.

FIG. 6 is a schematic depicting a retractor blade with a screw gate according to one embodiment of the present invention.

FIG. 7 is a diagram depicting the driving of a bone screw into the pedicle according to one embodiment of the present invention.

FIG. 8 is a diagram depicting the securing of a pair of retractor blades into adjacent pedicles according to one embodiment of the present invention.

FIG. 9 is a diagram depicting the pair of retractor blades secured to adjacent pedicles according to one embodiment of the present invention.

FIG. 9 is a diagram depicting according to one embodiment of the present invention.

FIG. 10 is a schematic depicting various components of a retractor blade assembly according to one embodiment of the present invention.

FIG. 11 is a schematic depicting a retractor blade in which a bone screw is captured by the screw gate according to one embodiment of the present invention.

FIG. 12 is a schematic depicting a surgical retractor for use with the retractor blades of the present invention.

FIG. 13 is a flowchart depicting a method for connecting a retractor blade to a bone screw, according to one embodiment of the present invention.

### DETAILED DESCRIPTION OF THE INVENTION

Embodiments of the disclosure are generally directed to intervertebral implant devices. One or more fixation members, such as screws, nails, shims, tangs, spikes, staples, pins, fins, blades, or the like, may be used to secure the implant devices to adjacent vertebrae. The fixation members may also include a combination of these to provide for optimal ease of insertion and fixation of the device. The intervertebral implant devices may be included with an inserter for placing the implant devices into the intervertebral space and securing the implant devices to the vertebrae with the fixation members.

The embodiments of the disclosure and the various features and advantageous details thereof are explained more fully with reference to the non-limiting embodiments and examples that are described and/or illustrated in the accompanying drawings and detailed in the following description. The features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

Terms such as distal and proximal are used herein to designate the relative positions of components in relation to a particular view of the object designated, with proximal representing a part of the object closer and distal representing a part of the object farther removed from the proximal. These terms are used for clarity in description, but the examples and embodiments herein should not be construed to mean that the invention does not cover the same objects when viewed from a different perspective. Furthermore, in some cases (but not all), these terms may be synonymous with human body reference terms anterior and posterior.

One aspect of the present invention is related to preparing and securing a surgical pathway for operational procedures associated with the spine. The following description describes a typical procedure in the context of the present invention.

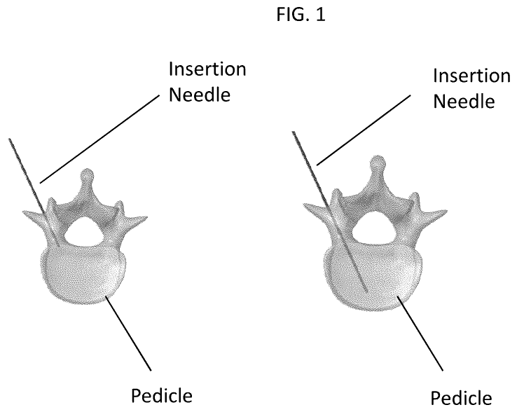

Preparation of the Pedicle: To begin the surgical procedures, a skin incision is made, and an insertion needle is placed through the skin incision to the intersection of the pedicle bone facet. FIG. 1 is a diagram depicting the insertion needle approach to and entry into the pedicle bone facet. In this way, the insertion needle gains access to the pedicle. After placing the insertion needle at the intersection of the pedicle bone facet, the needle may be advanced partially through the pedicle using for example by impacting the needle with a slap hammer. When the insertion needle reaches the medial wall of the pedicle (in the anterior/posterior view), verification is typically performed in the lateral view to ensure that the insertion needle is past the base of the pedicle. At this time, with the needle in a proper position, the inner trocar of the insertion needle is removed. The removal of the inner trocar allows a “KWire” to be inserted into the pedicle. Once the KWire is inserted, the outer shaft of the insertion needle is removed while holding the K-Wire in position.

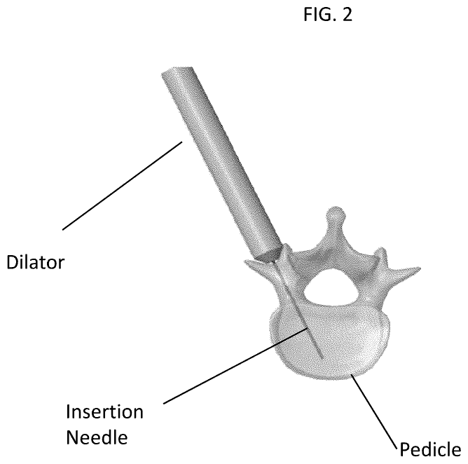

Dilation: Through the incision, place the smallest dilator over the K-Wire. FIG. 2 is a diagram depicting the placement of a dilator over a K-wire. Next, advance the smallest dilator over the K-Wire through the tissue twisting while directing it toward the pedicle. Location of the smallest dilator can be confirmed using imaging. Sequentially slide dilators from smallest to largest over each dilator until desired dilator is seated. Remove the earlier inserted smaller dilators while leaving the largest dilator in place as a working channel for pedicle preparation.

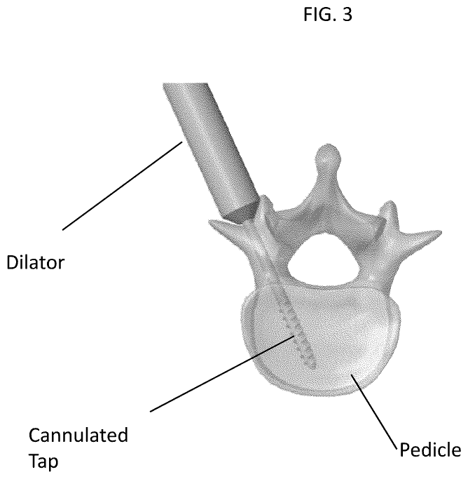

Tapping the Pedicle: A cannulated tap may be used to provide threads in the pedicle canal. FIG. 3 is a diagram depicting a cannulated tap inserted into the pedicle canal. The tap diameter of the cannulated tap is chosen according to the diameter of the bone screws to be implanted. Once an appropriate cannulated tap is selected, a cannulated tap is assembled to a ratcheting straight handle or to a ratcheting T-handle, and inserted over the K-Wire in the prepared dilated pathway. The cannulated tap is screwed down the desired length. At this point in time, the bone screws can be screwed into the pedicle.

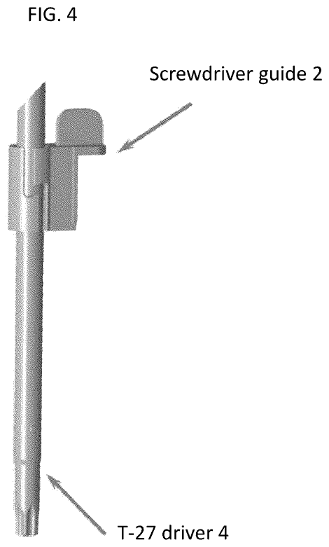

Screw & Blade Assembly: FIG. 4 is a schematic depicting a screwdriver guide assembly according to one embodiment of the present invention. With reference to FIG. 4, a screwdriver guide 2 is assembled to a screwdriver 4 by sliding guide 2 down the shaft of the handle end of screwdriver 4. Afterwards a handle is attached.

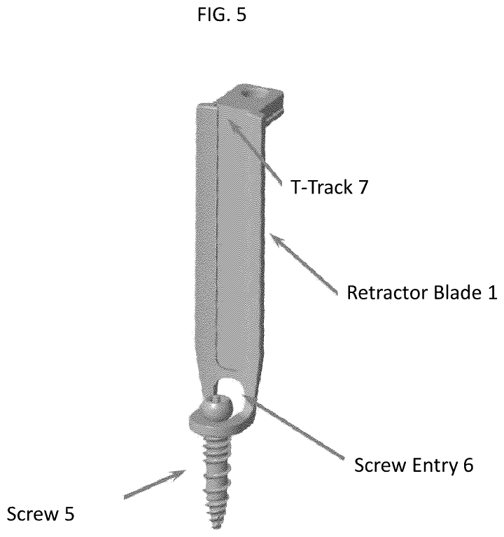

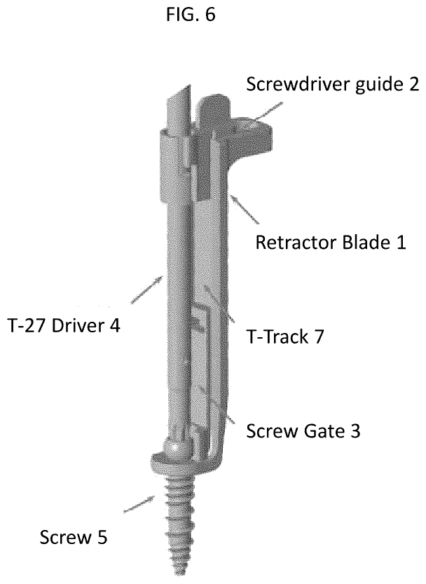

Next, an appropriately-sized bone screw 5 can be placed through opening on lower back 6 of retractor blade 1. FIG. 5 is a schematic depicting a retractor blade according to one embodiment of the present invention. The retractor blade 1 has a T-track 7 to be detailed below. With reference to FIG. 6, while holding bone screw 5 in place, screw gate 3 is slid down the T-track 7 of blade 1 until it stops at the bottom of the blade 1. Driver 4 is mated to screw 5 by a screwdriver guide 2 while sliding guide down T-track 7 of blade 1 until a stop is reached.

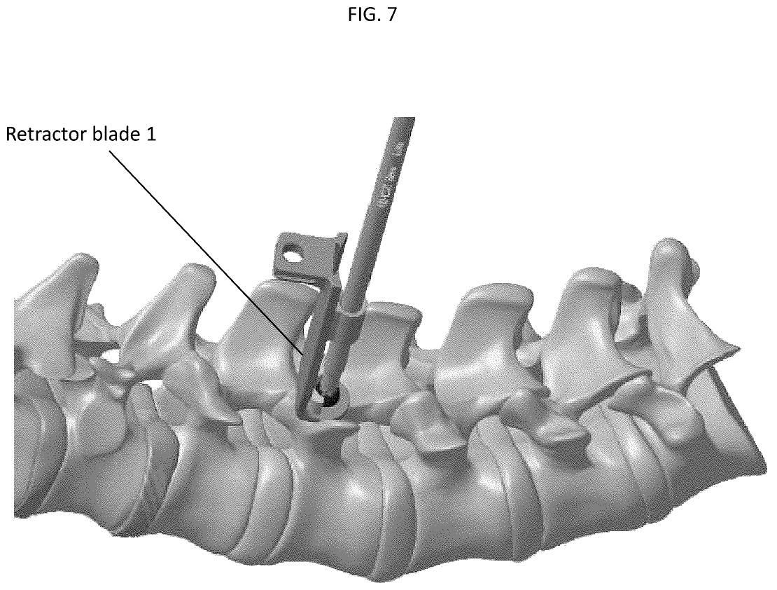

Retractor Blade/Pedicle Screw Insertion: In one embodiment of the present invention, the screw/driver/blade assembly shown in FIG. 6 is used to insert a bone screw into the pedicle. As shown in FIG. 7, a ratcheting handle on T-27 driver 4 is used to rotate bone screw 5 and thereby advance the bone screw 5 into the pedicle. The bone screw 5 is advanced into the vertebral body (the pedicle) until the last thread is flush with the bony surface, and retractor blade is seated on a surface of pedicle, as shown in FIG. 7.

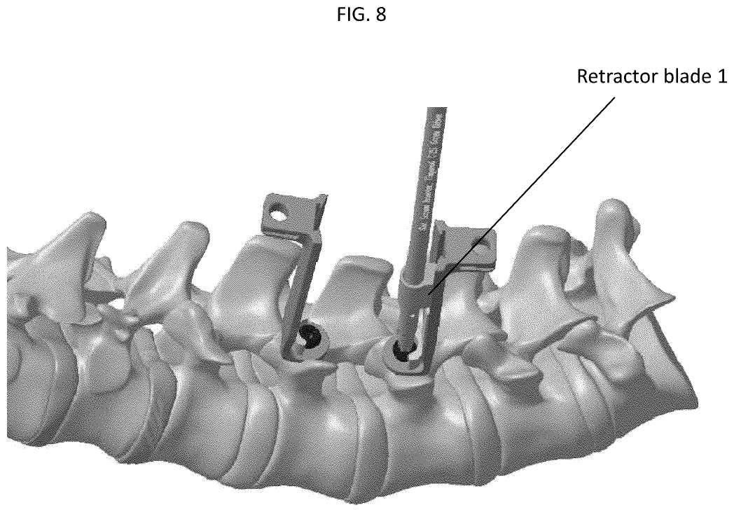

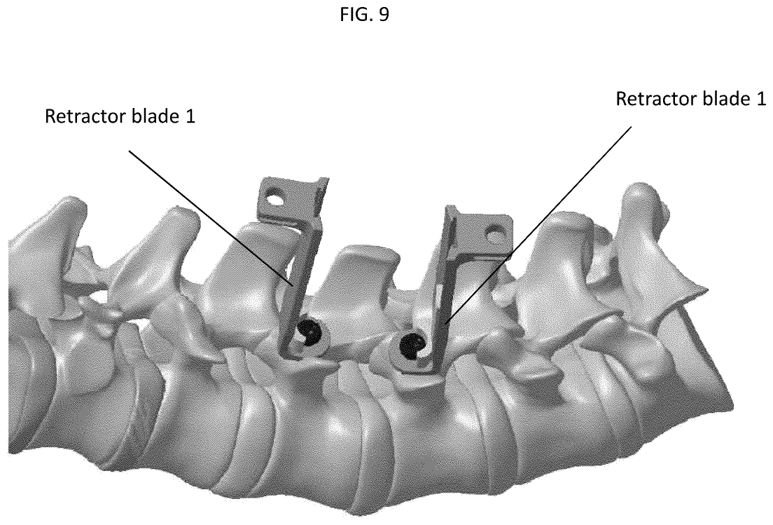

Next, the screwdriver guide is lifted from retractor blade 1 to disengage screwdriver from screw, and process above is repeated with a second retractor blade 1 and second bone screw 5 insertion. FIGS. 8 and 9 show the installation of a pair of retractor blades into adjacent pedicles.

Next, the screwdriver guide is lifted from second retractor blade 1, and fluoroscopic imaging can be used to confirm placement.

At this point, the retractor blades can be attached to a retractor for distraction of the pedicle and disc space preparation (as detailed below).

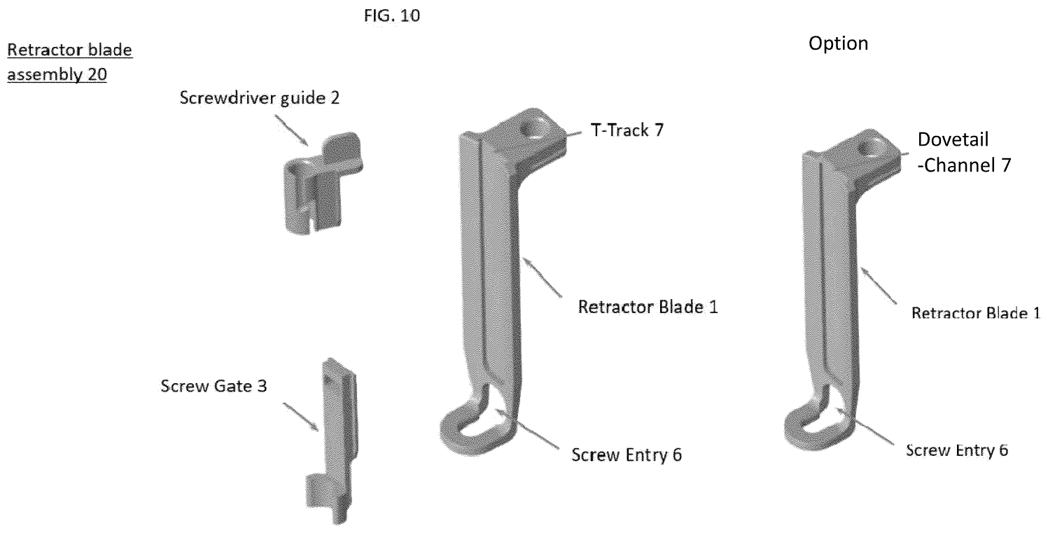

Retractor blade assembly: In one embodiment of the present invention, there is provided a retractor blade assembly as detailed above. The components of the retractor blade assembly 20 is shown in detail in FIG. 10. FIG. 10 is a schematic depicting components of a retractor blade assembly according to one embodiment of the present invention.

The components include retractor blade 1 having a track 7 (e.g. a T-track) extending longitudinally along a face of the blade for example until a stop at the bottom of the track is reached. Retractor blade 1 has (at the bottom) a screw entry 6 comprising an opening or window through which a bone screw is insertable. Retractor blade 1 has a screw gate 3 having a protrusion on a back surface which engages track 7. Once a bone screw is placed through screw entry 6 onto the blade 1, screw gate 3 can be slid down along track 7 until screw gate 3 blocks the screw entry 6 for example by covering or partially covering the opening. In one embodiment of the present invention, the screw gate (once slid down track 7) may be locked in position and cannot readily move back up track 7. FIG. 10 shows an option where retractor blade 1 would have a dovetail channel rather than a T-channel. In this option, the protrusion on the back of screw gate 3 would be designed to slide in the dovetail channel.

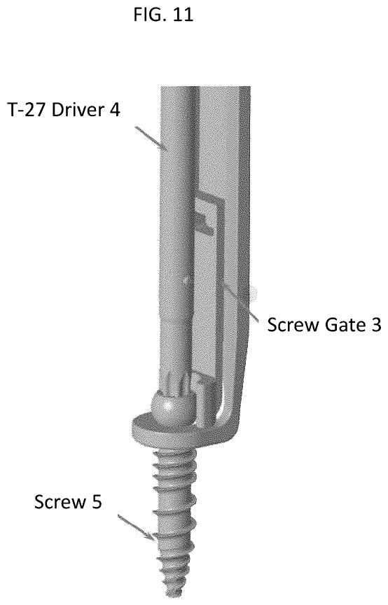

At this point in time, the driver 4 and screwdriver guide 2 (shown in FIG. 6) can be attached. A detailed view of this assembly with the bone screw captured by the screw gate 3 and the driver engaged with bone screw 5 is shown in FIG. 11.

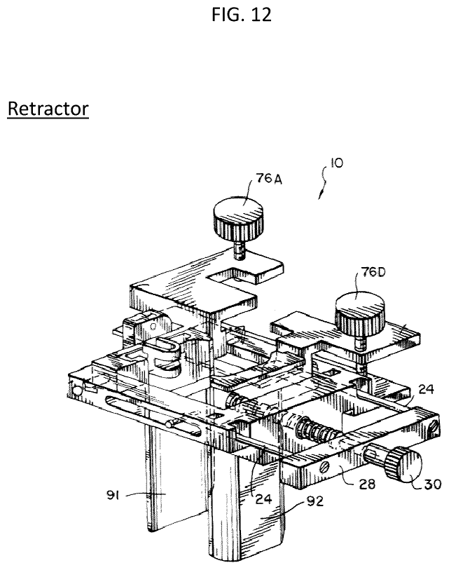

Retractor: As noted above, retractor blades once having been secured by the screw gate can be attached to retractor for distraction of the pedicle and disc space preparation. The present invention is not limited to a particular kind of retractor. For the purpose of illustration, the retractor of U.S. Pat. No. 6,224,545 (the entire contents of which are incorporated herein by reference) is shown below in FIG. 12. Here, in the present invention, the screw gate and blades described above would replace one or more of the retractor blades 91, 92 shown below in FIG. 12.

With the retractor 10 of FIG. 12, traveler rods 24 attached at or adjacent to opposed ends of thread assembly crosspiece 28. Turning knob 30 directly turns a threaded rod to adjust the spacing between a proximal end of first blade 91 and a proximal end of second blade 92. Moreover, by turning the thumbscrews 76A, 76D a surgeon can achieve an operating window within the patient for example which can be up to 8 inches larger than the operating window at the surface of the patient's skin because each distal end of blades 91 and 92 can move outwardly away from each other a distance of as much as about 4 inches. This makes possible a wide variety of surgeries with only a minimal incision.

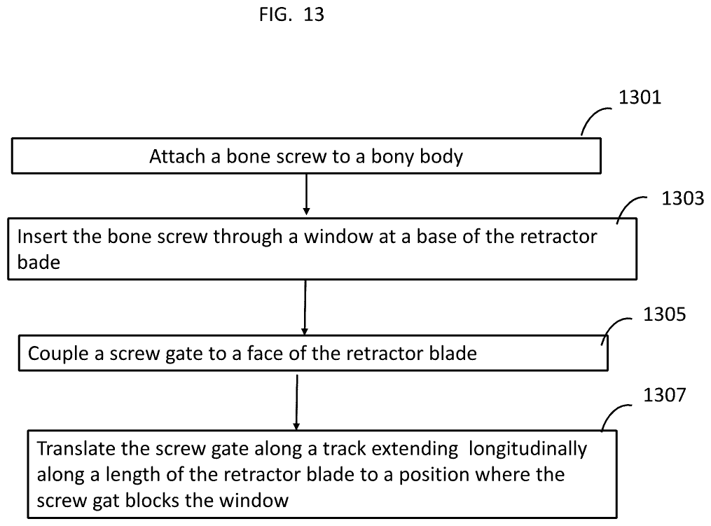

Method of Operation: In one embodiment, there is provided a method for connecting a retractor blade to a bone screw. FIG. 13 is a flowchart depicting this method. In this method, at 1301, a bone screw is attached to a bony body. At 1303 the bone screw is inserted through a window at a base of the retractor bade. At 1305, a screw gate is coupled to a face of the retractor blade. At 1307, screw gate is translated along a longitudinal length of the retractor blade to a position where the screw gate blocks the window.

This invention is also not limited to the type of material that the retractor device is made of. The retractor devices of the present invention can be made of any material appropriate for human implantation and having the mechanical properties sufficient to be utilized for the intended purpose of spinal fusion, including various metals such as cobalt chrome, stainless steel or titanium including its alloys, various plastics including those which are bio-absorbable, and various ceramics or combination sufficient for the intended purpose.

This invention is also not limited to the methods by which retractor devices are made. The individual components can be machined from solid stock pieces. Molding can be used to make the individual components. In this case, machining to final dimensions may or may not be in order. The surfaces once properly dimensioned can be coated with a variety of biocompatible coatings and/or surface treatments.

In some embodiments, any of the retractor devices described above can be used with additional implants and instruments. In some embodiments, the implants and instruments can be used with stabilization members, such as plates, screws, and rods.

Numerous modifications and variations of the present invention are possible in light of the above teachings. It is therefore to be understood that within the scope of the appended claims, the invention may be practiced otherwise than as specifically described herein.

1. A retractor blade assembly comprising:
a retractor blade having a track extending in a longitudinal direction along on a face of the retractor blade;
a screw entry at a bottom of the retractor blade comprising a window through which a bone screw is insertable; and
a screw gate having a protrusion on a back surface which engages the track on the retractor blade,
wherein, once the bone screw is placed through the screw entry, the screw gate lowers until the screw gate blocks the window.

2. The assembly of claim 1, wherein the track which translates the screw gate comprises a T channel, and the protrusion slides inside the T channel.

3. The assembly of claim 1, wherein the track which translates the screw gate comprises a dovetail channel, and the protrusion slides inside the dovetail channel.

4. The assembly of claim 1, wherein the window in the retractor blade forms an opening that extends along the base of the retractor blade and extends away from the base in the longitudinal direction.

5. The assembly of claim 1, further comprising a screwdriver guide for holding a screwdriver in position while advancing the bone screw into a bony body.

6. The assembly of claim 1, wherein the screwdriver guide is slidable in the track of the retractor blade.

7. The assembly of claim 1, wherein the retractor blade has a fixture on a top which attaches to a retractor arm.

8. The assembly of claim 1, wherein the screw gate has a base comprising a contoured surface partially encircling a head of the bone screw when the screw gate blocks the window.

9. The assembly of claim 8, wherein the base completely blocks the window.

10. The assembly of claim 8, wherein the base partially blocks the window.

11. A method for connecting a retractor blade to a bone screw, comprising:
attaching the bone screw to a bony body;
inserting the bone screw through a window at a base of the retractor bade;
coupling a screw gate to a face of the retractor blade; and
translating the screw gate along a track extending longitudinally along a length of the retractor blade to a position where the screw gat blocks the window.

12. The method of claim 11, wherein the track which translates the screw gate comprises a T channel, and the protrusion slides inside the T channel.

13. The method of claim 11, wherein the track which translates the screw gate comprises a dovetail channel, and the protrusion slides inside the dovetail channel.

14. The method of claim 11, wherein the window in the retractor blade forms an opening that extends along the base of the retractor blade and extends away from the base in the longitudinal direction.

15. The method of claim 11, further comprising holding a screwdriver in position with a screwdriver guide while advancing the bone screw into a bony body.

16. The method of claim 11, wherein the screwdriver guide is slidable in the track of the retractor blade.

17. The method of claim 11, further comprising attaching the retractor blade to a retractor arm.

18. The method of claim 11, further comprising partially encircling a head of the bone screw when the screw gate blocks the window.

19. The method of claim 18, wherein the partially encircling completely blocks the window.

20. The method of claim 18, wherein the partially encircling partially blocks the window.
